(19) United States
(12) Patent Application Publication
(10) Pub. No.: US 2008/0242609 A1
(43) Pub. Date: Oct. 2, 2008

(54) Composition for the therapy of diabetes mellitus and adiposity

(75) Inventors: Wolf Georg Forssmann, Hannover (DE); Rudolf Richter, Hannover (DE); Knut Adermann, Hannover (DE); Markus Meyer, Hannover (DE)

Correspondence Address: JACOBSON HOLMAN PLLC, 400 SEVENTH STREET N.W., SUITE 600, WASHINGTON, DC, 20004, US

(73) Assignee: Pharis Biotec GmbH.

(21) Appl. No.: 11/907,623

(22) Filed: Oct. 15, 2007

Related U.S. Application Data
(63) Continuation of application No. 10/844,598, filed on May 13, 2004, now abandoned.
Application No. 10/844,598 is a continuation of application No. 09/508,083, filed on Jul. 3, 2000, now abandoned.

(30) Foreign Application Priority Data
Sep. 12, 1997 (DE) 197 40 081.7
Dec. 23, 1997 (DE) 197 57 739.3
Mar. 11, 1998 (DE) 198 10 515.0

(51) Int. Cl. A61K 38/17; C07K 14/47; A61P 3/10

(52) U.S. Cl. 514/12; 530/324

(57) ABSTRACT

A composition containing at least two of the following active substances A, B, C, wherein:

A=at least one hormone stimulating the production of cAMP;

B=at least one substance inhibiting the degradation of a cyclic nucleotide;

C=at least one hormone stimulating the production of cGMP.

The present invention relates to the composition of claims 1 to 29 and a medicament containing the compositions according to the invention.

The hormonal regulation of blood sugar homeostasis is effected primarily by the pancreatic hormones insulin, glucagon and somatostatin. They are produced in the islets of Langerhans in the pancreas. This endocrine regulation of the blood sugar level is in turn under the complex control by metabolites (glucose, amino acids, catecholamines, etc.) circulating with the blood. Although insulin secretion from the endocrine pancreas is predominantly stimulated by the blood glucose level, there are also paracrine factors in the form of hormones, such as glucagon and somatostatin, which affect insulin secretion. The modulation of insulin secretion in the islet cells of the pancreas is mediated by the second messenger cyclic adenosine monophosphate (cAMP).

The CAMP metabolism of the islet cells of the pancreas is regulated on different levels. On the one hand, the production of CAMP can be stimulated in the pancreatic beta cells, and on the other hand, the degradation of CAMP in the pancreatic beta cells can be stimulated or inhibited by various phosphodiesterases.

Phosphodiesterases are enzymes which degrade cyclic nucleotides (CAMP, cGMP). Today, a distinction is made between seven different groups of phosphodiesterases which possess different substrate specificities and/or different mechanisms of activation/inhibition. For the different groups of phosphodiesterases, specific inhibitors have been described (for example: PDE I inhibitor: vinpocetin; PDE II inhibitor: trequinsin; PDE III inhibitor: milrinone; PDE IV inhibitor: rolipram; PDE V inhibitor: zaprinast).

Guanylin and uroguanylin are peptide hormones formed in the intestine which circulate in the blood. They belong to the guanylate cyclase activating peptides and stimulate the formation of cyclic guanosine monophosphate in various tissues.

Surprisingly, it has been found that a composition containing at least two of the following active substances A, B, C, wherein:

A=at least one hormone stimulating the production of CAMP;

B=at least one substance inhibiting the degradation of a cyclic nucleotide;

C=at least one hormone stimulating the production of cGMP;

is superior in therapy to the administration of the individual active substances.

The active substance A, for example, is a GLP-1/GLP-1-like peptide, preferably GLP-1(7-34)-amide and/or GLP-1(7-36)-amide. Surprisingly, the native plasma form GLP-1(7-34)-COOH and GLP-1(7-34)-amide have a half life which is twice to three times longer than that of GLP-1(7-36)-amide.

Further, infusion of GLP-1(7-34)-COOH and. GLP-1(7-34)-amide in equimolar amounts results in a significantly higher insulin release and a significantly higher reduction of the glucose level than the infusion of GLP-1(7-36)-amide does.

The active substance B, for example, is a phosphodiesterase inhibitor, preferably a group III and/or IV phosphodiesterase inhibitor.

Active substance C, for example, is a guanylate cyclase C activating peptide from the guanylin and/or uroguanylin genes, preferably guanylin-101-115 and/or uroguanylin-89-112.

The composition according to the invention can be employed in combination with one or more peptide hormones which affect the islet cell secretion, such as the hormones of the secretin/gastric inhibitory peptide.(GIP)/vaso-active intestinal peptide (VIP)/pituitary adenylate cyclase activating peptide (PACAP)/glucagon-like peptide II (GLP-II)/glicentin/glucagon gene family and/or those of the adrenomedullin/amylin/calcitonin gene related peptide (CGRP) gene family.

Preferably, the composition according to the invention is used with GLP-1 as GLP-1(7-34), GLP-1(7-35), GLP-1(7-36) or GLP-1(7-37) in its C-terminally carboxylated or amidated form or as modified GLP-1 peptides with the following modifications:

(a) substitution of the amino acid lysine in position 26 and/or 34 by a neutral amino acid, arginine or a D-form of lysine or arginine; and/or substitution of arginine in position 36 by a neutral amino acid, arginine or a D-form of arginine or lysine;

(b) substitution of tryptophan in position 31 by an oxidation-resistant amino acid;

(c) at least one substitution in the following positions by the respectively stated amino acid:

Y for V in position 16;

K for S in position 18;

D for E in position 21;

S for G in position 22;

R for Q in position 23;

R for A in position 24; and

Q for K in position 26;

(d) at least one substitution in the following positions by the respectively stated amino acid:

a small neutral amino acid for A in position 8;

an acidic or neutral amino acid for E in position 9;

a neutral amino acid for G in position 10; and

an acidic amino acid for D in position 15; and/or

(e) substitution of the amino acid histidine in position 7 by a neutral amino acid or the D-form or N-acetylated or N-alkylated form of histidine wherein the amino acids for the stated substitutions are in either the D- or L-form, and the amino acid substituted in position 7 is substituted in either its N-acetylated or its N-alkylated form.

In another preferred embodiment, the composition according to the invention contains modifications from the exchange of amino acids in the D- or L-form. In particular, those modifications are possible in which the amino acid lysine in positions 26 and/or 34 is substituted by K+, G, S, A, L, I, Q, M, R and R+, and the amino acid arginine in position 36 is substituted by K, K+, G, S, A, L, I, Q, M and R+, and/or the amino acid tryptophan in position 31 is substituted by F, V, L, I, A and Y (the symbol t means the D-form of the corresponding amino acid).

Optionally, the modifications stated above may be combined with at least one of the substitutions S for G in position 22, R for Q and A in positions 23 and 24, and Q for K in position 26, or these substitutions may be additionally combined with a substitution of D for E in position 21.

Another modification is the substitution wherein alanine in position 8 is substituted by a small neutral amino acid from the group consisting of S, S+, G, C, Ct, Sar, A+, beta-ala and Aib, wherein the acidic or neutral amino acid substituted for glutamic acid in position 9 is selected from the group consisting of E+, D, D+, Cay, T, T+, N, N+, Q, Q+, Cit, MSO and acetyl-K, and wherein the neutral amino acid substituted for glycine in position 10 is selected from the group consisting of S, S+, Y, Y+, T, T+, N, N+, Q, Q+, Cit, MSO, acetyl-K, F and F+.

A modification wherein the amino acid substituted for histidine in position 7 is selected from the group consisting of H+, Y, Y+, F, F+, R, R+, Orn, Orn+, M, M+, N-formyl-H, N-formyl-H+, N-acetyl-H, N-acetyl-H+, N-isopropyl-H, N-isopropyl-H+, N-acetyl-K, N-acetyl-K+, P and P+ may also be used.

In particular, the following modified peptides may be used in the compositions according to the invention:

(H+)7-GLP-1(7-37), (Y)7-GLP-1(7-37), (N-acetyl-H)7-GLP-1(7-37), (N-iso-propyl-H)7-GLP-1(7-37), (A+)8-GLP-1(7-37), (E+)9-GLP-1(7-37), (D)9-GLP-1(7-37), (D+)9-GLP-1(7-37), (F+)10-GLP-1(7-37), (S)22(R)23(R)24 (Q)26-GLP-1(7-37), and/or (S)8(Q)9(Y)16(K)18(D)21-GLP-I(7-37).

Further, as the active substance A in the composition according to the invention, there may be used a peptide which has an increased resistance to degradation in the plasma as compared to GLP-1(7-34), GLP-1(7-35), GLP-1(7-36) or GLP-1(7-37)- or the C-terminal amide, and/or has at least one of the following modifications:

(α) substitution of histidine in position 7 by the D-form of a neutral or acidic amino acid or the D-form of histidine;

(β) substitution of alanine in position 8 by the D-form of an amino acid; and

(χ) substitution of histidine in position 7 by an N-acylated (1-6C) or N-alkylated (1-6C) form of an alternative amino acid or histidine.

Histidine in position 7 may be substituted by an amino acid from the group consisting of P+, D+, E+, N+, Q+, L+, V+, I+ and H+, the D-amino acid in position 8 may be substituted by an amino acid from the group consisting of P+, V+, L+, I+ and A+ and/or the D-amino acid in position 8 may be substituted by an alkylated or acetylated amino acid from the group consisting of P, D, E, N, Q, V, L, I, K, and H.

In another preferred embodiment, the composition according to the invention contains at least one modified peptide of the following type: (H+)7-GLP-1(7-37), (N-acetyl-H)7-GLP-1(7-37), (N-isopropyl-H)7-GLP-1(7-37), (N-acetyl-K)7-GLP-1(7-37) and/or (A+)8-GLP-1(7-37).

One skilled in the art will understand that the peptide active substances may be present in a phosphorylated, acetylated and/or glycosylated form;

In particular, those derivatives derived from GLP-1-(7-34)COOH and the corresponding acid amide are employed which have the following general formula:

R—NH—HAEGTFTSDVSSYLEGQAAKEFIAWLVK—CONH₂,

wherein R=H or an organic compound having from 1 to 10 carbon atoms. Preferably, R is the residue of a carboxylic acid. Particularly preferred are the following carboxylic acid residues: formyl, acetyl, propionyl, isopropionyl, methyl, ethyl, propyl, isopropyl, n-butyl, sec-butyl, tert-butyl.

As the active substance B in the composition according to the invention, there are employed, in particular, non-specific phosphodiesterase inhibitors, such as papaverine, theophylline, enprofyllines and/or IBMX, or specific phosphodiesterase inhibitors.

Particularly preferred are phosphodiesterase inhibitors which inhibit group III phosphodiesterases (cGMP-inhibited phosphodiesterases), such as indolidane (LY195115), cilostamide (OPC 3689), lixazinone (RS 82856), Y-590, imazodane (CI914), SKF 94120, quazinone, ICI 153,110, cilostazole, bemorandane (RWJ 22867), siguazodane (SK&F 94-836), adibendane (BM 14,478), milrinone (WIN 47203), enoximone (MDL 17043), pimobendane (UD-CG 115), MCI-154, saterinone (BDF 8634), sulmazole (ARL 115), UD-CG 212, motapizone, piroximone, ICI 118233, and/or phosphodiesterase inhibitors which inhibit group IV phosphodiesterases (cAMP-specific phosphodiesterases), such as rolipram ZK 62711; pyrrolidone), imidazolidinone (RO 20-1724), etazolate (SQ 65442), denbufylline (BRL 30892), ICI63197 and/or RP73401.

The phosphodiesterase inhibitors which can inhibit both group III and group IV phosphodiesterases, such as tolafentrine, zardaverine, EMD54622 and/or Org30029, can also be used in the composition according to the invention.

The medicament according to the invention contains an effective amount of the composition according to the invention and can be used for the therapy of insulin-dependent diabetes mellitus, non-insulin-dependent diabetes mellitus, MODY (maturity-onset diabetes in young people), secondary hyperglycemias in connection with pancreatic diseases (chronic pancreatitis, pancreasectomy, hemochromatosis) or endocrine diseases (acromegaly, Cushing's syndrome, pheochromocytoma or hyperthyreosis), drug-induced hyperglycemias (benzothiadiazine saluretics, diazoxide or glucocorticoids), pathologic glucose tolerance, hyperglycemias, dyslipoproteinemias, adiposity, hyperlipoproteinemias and/or hypotensions.

Surprisingly, the compositions according to the invention exhibit a significantly better therapeutical effect in diabetes mellitus, for example, than the monotherapies with the individual components.

Studies have shown that the compositions according to the invention lead to a significantly higher insulin release in animal experiments as compared to the individual components GLP-1, phosphodiesterase inhibitors, guanylin or uroguanylin. The blood sugar level is decreased by the composition according to the invention to a significantly higher extent than by the respective individual components. Further, it has been found that the therapeutic dosage of the compositions according to the invention, especially of GLP-1, could be significantly reduced. For the other components of the composition according to the invention, there is also a positive synergistic effect.

In animal experiments, it could be shown, surprisingly, that the duration of action of GLP-1 on the blood sugar level can be prolonged by a factor of 4 to 5 by combining it with phosphodiesterase or the guanylate cyclase activating peptides. These results are based on the determination of the blood sugar level upon one intravenous injection of the different combinations. Subsequently, the blood sugar level was determined over a period of 6 hours.

While GLP-1 must be continuously administered in a monotherapy, discontinuous delivery in a suitable dosage form can be achieved through the inventive combination with phosphodiesterases or guanylate cyclase activating peptides. Surprisingly, it has been found in the studies that the therapeutically effective GLP-1 dosage is lower by a power of ten in the combination therapies as compared to the monotherapy with GLP-1. The side effects of GLP-1 monotherapy, especially delayed stomach discharge, could be eliminated both by phosphodiesterase inhibitors and by guanylin or uroguanylin.

Surprisingly, after a single application of the combination therapy, not only is the postprandial rise of the blood sugar level reduced, but also a subsequent decrease of the glucose level to an almost normal blood sugar level is achieved.

This shows that a continuous delivery of GLP-1 can be dispensed with in the combination according to the invention.

The compositions according to the invention with the individual components GLP-1, phosphodiesterase inhibitors, guanylin or uroguanylin were examined in vitro in a bioactivity assay. In this cellular assay, the formation of CAMP is examined. The compositions according to the invention gave a significantly higher level of CAMP formation in the assay as compared to the individual components.

Surprisingly, it has been found in studies on the functional mechanism of the action of guanylin and uroguanylin on insulin secretion that cGMP analogues result in an increase of the CAMP concentration in the islet cells.

Surprisingly, administration of the composition according to the invention will prolong the duration of action of the individual components.

The compositions according to the invention reduce the need for insulin in diabetes mellitus to a higher extent than is achieved by a corresponding administration of individual components of the compositions according to the invention.

The compositions according to the invention are suitable for the therapy of insulin-dependent diabetes mellitus, non-insulin-dependent diabetes mellitus, MODY (maturity-onset diabetes in young people), secondary hyperglycemias in connection with pancreatic diseases (chronic pancreatitis, pancreasectomy, hemochromatosis) or endocrine diseases (acromegaly, Cushing's syndrome, pheochromocytoma or hyperthyreosis), drug-induced hyperglycemias (benzothiadiazine saluretics, diazoxide or glucocorticoids), pathologic glucose tolerance, hyperglycemias, dyslipoproteinemias, adiposity, hyperlipoproteinemias and/or hypotensions.

The compositions according to the invention can be employed together with peptide hormones which are structurally related to glucagon, and/or with the peptide hormones adrenomedullin, amylin and/or calcitonin gene related peptide (CGRP). The hormones belonging to the glucagon multigene family are secretin, gastric inhibitory peptide (GIP), vasoactive intestinal peptide (VIP), pituitary adenylate cyclase activating peptide (PACAP), glucagon-like peptide II (GLP-II) and glicentin. These peptides regulate glucose metabolism, gastro-intestinal mobility and secretory processing in different ways. All gene products of secretin GIP, VIP, PACAP, GLP-II, glicentin, adrenomedullin, amylin and CGRP as well as modified substances of secretin, GIP, VIP, PACAP, GLP-II, glicentin, adrenomedullin, amylin and CGRP can be used for such therapy.

For the therapy of diabetes mellitus or adiposity by the compositions according to the invention, GLP-1(7-34), GLP-1(7-35), GLP-1(7-36) or GLP-1(7-37) in its C-terminally carboxylated or amidated form or as modified GLP-1 peptides with higher biological activity can be used.

For the therapy of diabetes mellitus or adiposity using the compositions according to the invention, as the active substance B, there may be used non-specific phosphodiesterase inhibitors, such as papaverine, theophylline, enprofyllines and/or IBMX; and/or specific phosphodiesterase inhibitors and especially those phosphodiesterase inhibitors which inhibit group III phosphodiesterases (cGMP-inhibited phosphodiesterases), including indolidane (LY195115), cilostamide (OPC 3689), lixazinone (RS 82856), Y-590, imazodane (CI914), SKF 94120, quazinone, ICI 153,110, cilostazole, bemorandane (RWJ 22867), siguazodane (SK&F 94-836), adibendane (BM 14,478), milrinone (WIN 47203), enoximone (MDL 17043), pimobendane (UD-CG 115), MCI-154, saterinone (BDF 8634), sulmazole (ARL 115), UD-CG 212, motapizone, piroximone, ICI 118233.

Further, there may be used phosphodiesterase inhibitors which inhibit group IV phosphodiesterases (cAMP-specific phosphodiesterases), such as rolipram ZK 62711; pyrrolidone), imidazolidinone (RO 20-1724), etazolate (SQ 65442), denbufylline.(BRL 30892), ICI63197, RP73401.

Phosphodiesterase inhibitors which inhibit both group III and group IV phosphodiesterases, such as tolafentrine, zardaverine, EMD54622, Org30029, can also be used.

In vitro examinations on RIN cells surprisingly showed that specific PDE II and PDE IV inhibitors, in particular, inhibit the degradation of CAMP.

The combination of specific PDE II inhibitors and GLP-1-(7-34) induces a 5 to 10 times higher intracellular CAMP concentration as compared to administration of the individual substances. Further, it could be shown that the combination of specific PDE IV inhibitors and GLP-1-(7-34) induces a 10 to 15 times higher intracellular CAMP concentration as compared to administration of the individual substances.

As the active substance C, guanylate C activating peptides from the guanylin and/or uroguanylin genes, preferably guanylin-101-115 and/or uroguanylin-89-112, can be used.

For the therapy of diabetes mellitus or adiposity using the compositions according to the invention, the gene products of guanylin and uroguanylin or modified, more biologically active molecules of guanylin and/or uroguanylin may be employed.

The combination of specific PDE II inhibitors and guanylin induces a 2 to 3 times higher intracellular CAMP concentration as compared to the individual substance PDE II inhibitor and a 5 to 7 times higher intracellular CAMP concentration as compared to the individual substance guanylin. Further, it could be shown that the combination of specific PDE IV inhibitors and guanylin induces a 2 to 3 times higher intracellular CAMP concentration as compared to the individual substance PDE IV inhibitor and a 10 to 15 times higher intracellular CAMP concentration as compared to the individual substance guanylin.

Similarly, the pharmacologically acceptable salts are obtained by neutralization of the bases with inorganic or organic acids. As inorganic acids, there may be used, for example, hydrochloric, sulfuric, phosphoric or hydrobromic acid, and as organic acids, there may be used, for example, carboxylic, sulfo or sulfonic acids, such as acetic, tartaric, lactic, succinic, alginic, benzoic, 2-phenoxybenzoic, 2-acetoxybenzoic, cinnamic, mandelic, citric, malic, salicylic, 3-aminosalicylic, ascorbic, embonic, nicotinic, isonicotinic or oxalic acid, amino acids, methanesulfonic, ethanesulfonic, 2-hydroxy-ethanesulfonic, ethane-1,2-disulfonic, benzenesulfonic, 4-methylbenzene-sulfonic or naphthalene-2-sulfonic acid.

For the preparation of the medicaments for the treatment of the mentioned diseases, a therapeutically effective combination of the individual substances or their salts is used, in addition to the usual auxiliary agents, carriers and additives. The dosage of the combination preparation depends on the species, body weight, age, individual condition of the patient and the way of administration.

Peptide containing medicaments are prepared by the method known to those skilled in the art for suitable ways of administration. Thus, in particular, oral, intravenous, intramuscular, intracutaneous, intrathecal and transpulmonary administrations may be used. The dosage to be administered for GLP-1 and its analogues is preferably from 0.1 μg per kg of body weight to 10 mg per kg of body weight. The dosage to be administered for guanylin and its analogues is preferably from 0.1 μg per kg of body weight to 10 mg per kg of body weight. The dosage to be administered for uroguanylin and its analogues is preferably from 0.1 μg per kg of body weight to 10 mg per kg of body weight. The peptides packaged in micelles and biopolymers may also be used as dosage forms.

In addition, known release forms by which release from galenic dosage forms of the ingredients is achieved permanently or in a pulsatile way may also be used for administration. Preferably, they include biopolymers as carriers, liposomes as carriers or infusion pumps so that administration can be effected, inter alia, subcutaneously, intravenously, perorally, intramuscularly or transpulmonarily.

Solid dosage forms may contain inert auxiliary agents and carriers, such as calcium carbonate, calcium phosphate, sodium phosphate, lactulose, starch, mannitol, alginate, gelatin, guar gum, magnesium or aluminum stearate, methylcellulose, talcum, highly dispersed silicic acid, silicone oil, higher molecular weight fatty acids (such as stearic acid), agar-agar or vegetable or animal fats and oils, solid high molecular weight polymers (such as polyethylene glycol); formulations suitable for oral administration may also contain additional flavoring agents and/or sweeteners, if desired.

Liquid dosage forms may be sterilized and/or optionally contain auxiliary agents, such as preservatives, stabilizers, wetting agents, penetration agents, emulsifiers, spreading agents, solubilizers, salts for controlling the osmotic pressure or for buffering, and/or viscosity modifiers.

Such additives include, for example, tartrate and citrate buffers, ethanol, complexing (chelating) agents (such as ethylenediaminetetraacetic acid and its non-toxic salts). For controlling viscosity, there may be used high molecular weight polymers, such as liquid polyethylene oxide, carboxymethylcelluloses, polyvinylpyrrolidones, dextranes or gelatin. Solid carriers include, for example, starch, lactulose, mannitol, methylcellulose, talcum, highly dispersed silicic acid, higher molecular weight fatty acids (such as stearic acid), gelatin, agar-agar, calcium phosphate, magnesium stearate, animal and vegetable fats, solid high molecular weight polymers (such as polyethylene glycol).

Oily suspensions for parenteral applications may contain vegetable, synthetic or semisynthetic oils, such as liquid fatty acid esters having from 8 to 22 carbon atoms in the fatty acid chains, for example, palmitic, lauric, tridecanoic, margaric, stearic, arachic, myristic, behenic, pentadecanoic, linolic, elaidic, brassidic, erucic or oleic acid, esterified with mono- to trihydric alcohols having from 1 to 6 carbon atoms, such as methanol, ethanol, propanol, butanol, pentanol or their isomers, glycol or glycerol. Such fatty acid esters include, for example, commercial miglyols, isopropyl myristate, isopropyl palmitate, isopropyl stearate, PEG-6 caprate, caprylic/capric acid esters of saturated fatty alcohols, polyoxyethylene glycol trioleates, ethyl oleate, wax-like fatty acid esters, such as artificial duck uropygial gland fat, coconut oil fatty acid isopropyl ester, oleic acid oleyl ester, oleic acid decyl ester, lactic acid ethyl ester, dibutyl phthalate, adipic acid diisopropyl ester, polyol fatty acid ester, etc. Also suitable are silicone oils of different viscosities or fatty alcohols, such as isotridecyl alcohol, 2-octyidodecanol, cetylstearyl alcohol or oleyl alcohol, fatty acids, such as oleic acid. Further, vegetable oils, such as castor oil, almond oil, olive oil, sesame oil, cottonseed oil, peanut oil or soybean oil, may also be used.

As a solvent, gelling agent and solubilizer, there may be used water or water-miscible solvents. Suitable solvents include, for example, alcohols, such as ethanol or isopropyl alcohol, benzyl alcohol, 2-octyidodecanol, polyethylene glycol, waxes, methylcellosolve, cellosolve, esters, morpholine, dioxan, dimethyl sulfoxide, dimethylformamide, tetrahydrofuran, cyclohexane etc.

As film-forming agents, there may be used cellulose ethers which are soluble or swellable both in water and in organic solvents and, after drying, form a kind of film, such as hydroxypropylcellulose, methylcellulose, ethylcellulose or soluble starches. Thus, mixed forms between gelling agents and film-firming agents are also possible. Mainly, ionic macromolecules are employed, such as sodium carboxymethylcellulose, poly(acrylic acid), poly-(methacrylic acid) and their salts, sodium amylopectin semiglycolate, alginic acid propylene glycol alginate as the sodium salt, gum arabic, xanthane gum, guar gum or carrageen.

Further formulation aids that may be used include glycerol, paraffins of different viscosities, triethanolamine, collagen, allantoin, novantisolic acid, perfume oils.

The use of surfactants, emulsifiers or wetting agents may also be necessary for formulation, for example, sodium laurylsulfate, fatty alcohol ether sulfates, disodium N-lauryl-β-iminodipropionate, polyoxyethylated castor oil, or sorbitan monooleate, sorbitan monostearate, cetyl alcohol, lecithin, glycerol monostearate, polyethylene stearate, alkylphenol polyglycol ether, cetyltrimethylammonium chloride or mono-/dialkyl polyglycol ether orthophosphoric acid monoethanolamine salts.

Stabilizers, such as montmorillonite or colloidal silica, for the stabilization of emulsions or for preventing the degradation of the active substances, such as antioxidants, for example, tocopherols or butylhydroxyanisol, or preservatives, such as p-hydroxybenzoic acid ester, may also be necessary for preparing the desired formulations.

The manufacturing, filling and sealing of the preparations are performed under the usual antimicrobial and aseptic conditions. If possible, the preparations are packaged in separate unit doses for facilitating the handling; in this case too, as for the parenteral forms, if necessary for reasons of stability, the active substances or their combinations are separately packaged as a lyophilizate, optionally with solid carriers and the necessary solvents, etc.

### SEQUENCE LISTING

### (1) General Information:

(i) Applicant:
(A) Name: Prof. Dr. Wolf-Georg Forssmann
(B) Street: Feodor-Lynen-Str. 31
(C) City: Hannover
(E) Country: Germany
(F) Postal code: 30625

(ii) Title of invention: Composition for the therapy of diabetes mellitus and adiposity

(iii) Number of sequences: 1

(iv) Computer readable form:
(A) Medium type: Floppy disk
(B) Computer: IBM PC compatible
(C) Operating system: PC-DOS/MS-DOS
(D) Software: Patent In Release #1.0, Version #1.30 (EPO)

### (2) Information for SEQ ID NO: 1:

(i) Sequence characteristics:
(A) Length:28 amino acids
(B) Type: amino acid
(C) Strandedness: unknown
(D) Topology: unknown

(ii) Type of molecule: peptide

(xi) Sequence description: SEQ ID NO: 1:

His Ala Glu Gly Thr Phe Thr Ser Asp Val Ser Ser
1 5 10
Tyr Leu Glu Gly Gln Ala Ala Lys Glu Phe Ile Ala
15 20
Trp Leu Val Lys
25

1-33. (canceled)

34. A compound of the general formula
R—NH—HAEGTFTSDVSSYLEGQAAKEFIAWLVK—CONH₂ (SEQ ID NO: 1)
wherein R is acetyl.

35. The compound according to claim 34, wherein the compound exists in a phosphorylated, acetylated, and/or glycosylated form.

36. A method of using a pharmaceutical composition containing a compound according to claim 34, comprising administering the composition to a person for the treatment of insulin-independent diabetes mellitus.

37. The method according to claim 36, wherein the composition is administered in a long-lasting or pulsatile manner.

38. The method according to claim 36, wherein the composition is administered subcutaneously, intravenously, perorally, intramuscularly, or transpulmonarily.

39. A composition for human administration comprising the compound according to claim 34, in combination with a physiologically acceptable carrier or diluent.

40. The composition according to claim 39, in a release form by which the release of the compound is attained in a long-lasting or pulsatile manner.

41. The composition according to claim 39, suitable for subcutaneous, intravenous, or intramuscular administration.

42. The composition according to claim 39, suitable for peroral administration.

43. The composition according to claim 39, suitable for transpulmonary administration.

44. A composition comprising a therapeutically effective amount of the compound according to claim 35, in combination with a pharmaceutically acceptable carrier or diluent.

45. The composition according to claim 44, in a release form by which release of the compound is attained in a long-lasting or pulsatile manner.

46. The composition according to claim 44, suitable for subcutaneous, intravenous, or intramuscular administration.

47. The composition according to claim 44, suitable for peroral administration.

48. The composition according to claim 44, suitable for transpulmonary administration.


SEQUENCE LISTING (1 sequence)

SEQ ID NO: 1 - protein, 28 aa, Homo sapiens
HAEGTFTSDVSSYLEGQAAKEFIAWLVK